(19) United States
(12) Patent Application Publication
(10) Pub. No.: US 2010/0262183 A1
(43) Pub. Date: Oct. 14, 2010

(54) SYSTEMS DEVICES AND METHODS FOR ACHIEVING TRANSVERSE ORIENTATION IN THE TREATMENT OF SEPTAL DEFECTS

(75) Inventors: Ryan Abbott, San Jose, CA (US); W. Martin Belef, San Jose, CA (US); Dean Carson, Mountain View, CA (US); Ronald J. Jabba, Redwood City, CA (US); Anthony J. Pantages, San Jose, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: Ovalis Inc., Mountain View, CA (US)

(21) Appl. No.: 12/740,667

(22) PCT Filed: Nov. 5, 2008
(86) PCT No.: PCT/US08/82522

§ 371 Date: Apr. 29, 2010

Related U.S. Application Data
(60) Provisional application No. 60/986,229, filed on Nov. 7, 2007.

(51) Int. Cl. A61B 17/03

(52) U.S. Cl. 606/213

(57) ABSTRACT

Systems, devices and methods for treating internal tissue defects, such as septal defects, with implantable devices are provided. Elongate delivery systems for these implantable devices are configured to achieve a transverse orientation with respect to the defect site, allowing the implantable apparatus to be delivered from an elongate device in an efficient manner.

### BACKGROUND OF THE INVENTION

This application claims priority to U.S. Provisional Patent Application Ser. No. 60/986,229, which was filed on Nov. 7, 2007 and is fully incorporated by reference herein.

### FIELD OF THE INVENTION

The inventions described herein relate generally to the treatment of septal defects and more particularly, to systems, devices and methods for achieving generally transverse orientation of those systems and devices with respect to a defect in a septal wall.

Various defects can occur in the inter-atrial and inter-ventricular septal walls of the heart. For instance, abnormal openings in the inter-atrial septal wall can allow blood to shunt between the left and right atria. Inter-atrial defects can be generally classified as atrial septal defects (ASDs) or patent foramen ovales (PFOs). An ASD is generally defined as a direct opening in the septal wall that can allow blood to flow relatively unobstructed between the left and right atria. A PFO is generally defined as an opening existing between two flaps of atrial septal tissue, referred to as the septum primum and the septum secundum. Between the left and right ventricles, other septal defects known as ventricular septal defects (VSDs) can exist, which are generally defined as direct openings in the ventricular septal wall that can allow blood to flow relatively unobstructed between the left and right ventricles. Another type of cardiac defect, which is generally grouped together with the aforementioned septal defects, is a patent ductus arteriosus (PDA), which is an abnormal shunt between the aorta and pulmonary artery.

Treatment of these defects can be accomplished through direct surgical methods such as open-heart surgery, relatively less invasive trans-thoracic surgical methods and percutaneous intravascular methods using a catheter and the like. Of these, percutaneous intravascular methods are generally the most desirable because they require only a minor, remote surgical procedure (e.g., percutaneous access to a peripheral vein or artery, typically the femoral artery or vein) and thus avoid the increased risk, cost and recovery time that are associated with the more invasive approaches.

With an intravascular technique, access to the septal defect is least invasive if it takes place through one of the patient's already existing vessels leading to the desired cardiac chamber. Depending on the location of the defect and the cardiac chamber through which treatment will be administered, this approach can vary and, oftentimes, results in a less than optimal orientation of the treatment device with respect to the defect. For instance, access to the right atrial chamber for purposes of treating an ASD or PFO typically occurs through the superior vena cava (SVC) or inferior vena cava (IVC) (see FIG. 1E, which depicts an exemplary IVC approach). While the SVC and NC provide access, this route leaves the treatment device in a generally parallel, i.e., non-transverse, orientation to the septal wall, with the distal end of the device facing away from the septal wall. This can make the administration of treatments from the distal end of the device more difficult, especially if those treatments require the distal end of the device to be oriented such that it faces the septal wall.

Accordingly, improved systems, devices and methods for providing transverse orientation to a septal wall are needed.

### SUMMARY

Provided herein are systems, devices and methods configured to treat septal defects and other internal tissue defects by achieving a transverse orientation to the septal wall. These systems, devices and methods are provided by way of exemplary embodiments that should not be construed as limiting the systems and methods in any way.

Other systems, methods, features and advantages of the invention will be or will become apparent to one with skill in the art upon examination of the following figures and detailed description. It is intended that all such additional systems, methods, features and advantages be included within this description, be within the scope of the subject matter described herein, and be protected by the accompanying claims. In no way should the features of the exemplary embodiments be construed as limiting the appended claims absent express recitation of those features in the claims.

### BRIEF DESCRIPTION OF THE FIGURES

The details of the invention, both as to its structure and operation, may be gleaned in part by study of the accompanying figures, in which like reference numerals refer to like parts. The components in the figures are not necessarily to scale, emphasis instead being placed upon illustrating the principles of the invention. Moreover, all illustrations are intended to convey concepts, where relative sizes, shapes and other detailed attributes may be illustrated schematically rather than literally or precisely.

FIG. 1A is an exterior/interior view depicting an example human heart.

FIG. 1B is an enlarged side view of the septal wall depicting a PFO taken from the right atrium.

FIG. 1C is an enlarged side view of the septal wall depicting a PFO taken from the left atrium.

FIG. 1D is a cross-sectional view depicting an example PFO region taken along line 1D-1D of FIGS. 1B-C.

FIG. 1E is a perspective view depicting an exemplary catheter apparatus after advancement into a heart.

FIG. 2A is a side view depicting an exemplary embodiment of a treatment system.

FIG. 2B is a partial cross-sectional view depicting another exemplary embodiment of a treatment system during a procedure.

FIG. 2C is a cross-sectional view depicting an exemplary embodiment of a delivery member.

FIG. 2D is a partial cross-sectional view depicting an exemplary embodiment of a closure device after implantation within a septal wall.

FIGS. 2E-F are side views depicting exemplary embodiments of a piercing member.

FIG. 3A is a cross-sectional view depicting another exemplary embodiment of a delivery member.

FIG. 3B is a partial cross-sectional view depicting another exemplary embodiment of a treatment system during a procedure.

FIG. 3C is a perspective view depicting another exemplary embodiment of a treatment system.

FIG. 3D is a partial cross-sectional view depicting another exemplary embodiment of a treatment system during a procedure.

FIG. 4A is a side view depicting another exemplary embodiment of a treatment system.

FIGS. 4B-C are partial cross-sectional views depicting another exemplary embodiment of a treatment system during a procedure.

FIGS. 5A-C are side views depicting another exemplary embodiment of a treatment system.

FIG. 6A is a partial cross-sectional view depicting another exemplary embodiment of a treatment system during a procedure.

FIG. 6B is a cross-sectional view of region 6B of FIG. 6A.

FIG. 6C is a partial cross-sectional view depicting another exemplary embodiment of a treatment system.

FIG. 7A is a cross-sectional view depicting another exemplary embodiment of a delivery member.

FIG. 7B is a side view depicting another exemplary embodiment of a treatment system.

FIGS. 7C-D are partial cross-sectional views depicting another exemplary embodiment of a treatment system during a procedure.

FIGS. 8A-B are cross-sectional views depicting another exemplary embodiment of a treatment system.

FIG. 9A is a cross-sectional view depicting another exemplary embodiment of a treatment system.

FIGS. 9B-9C are partial cross-sectional views depicting additional exemplary embodiments of a treatment system during a procedure.

FIG. 9D is a side view depicting an additional exemplary embodiment of a treatment system.

FIG. 9E is a partial cross-sectional view depicting an additional exemplary embodiment of a treatment system during a procedure.

FIGS. 9F-G are cross-sectional views depicting additional exemplary embodiments of an implant within a septal wall.

FIGS. 10A-B are partial cross-sectional views depicting additional exemplary embodiments of a treatment system during a procedure.

FIG. 11 is a side view depicting another exemplary embodiment of a delivery member.

FIG. 12 is a partial cross-sectional view depicting an additional exemplary embodiment of a treatment system during a procedure.

### DETAILED DESCRIPTION

Provided herein are systems, devices and methods for treating septal defects using a percutaneous intravascular technique. For ease of discussion, these systems, devices and methods will be described with reference to treatment of a PFO. However, it should be understood that these devices, systems and methods can be used in treatment of any type of septal defect including ASD's, VSD's and the like, as well as PDA's, pulmonary shunts or other structural cardiac or vascular defects or non-vascular defects, and also any other tissue defect including non-septal tissue defects.

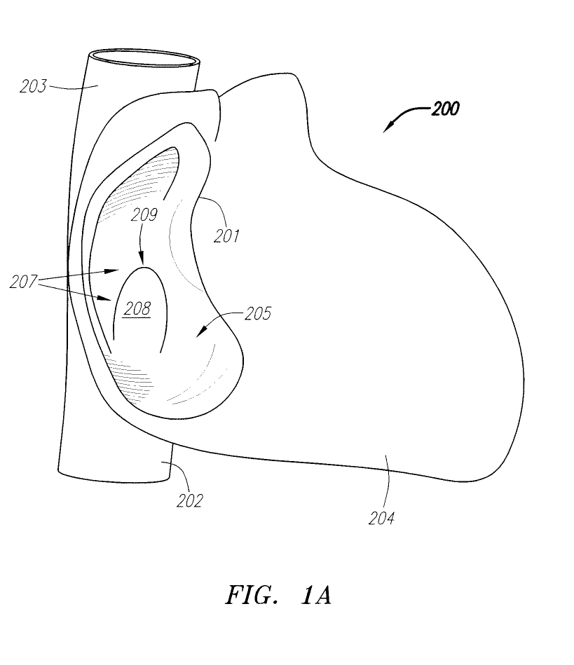

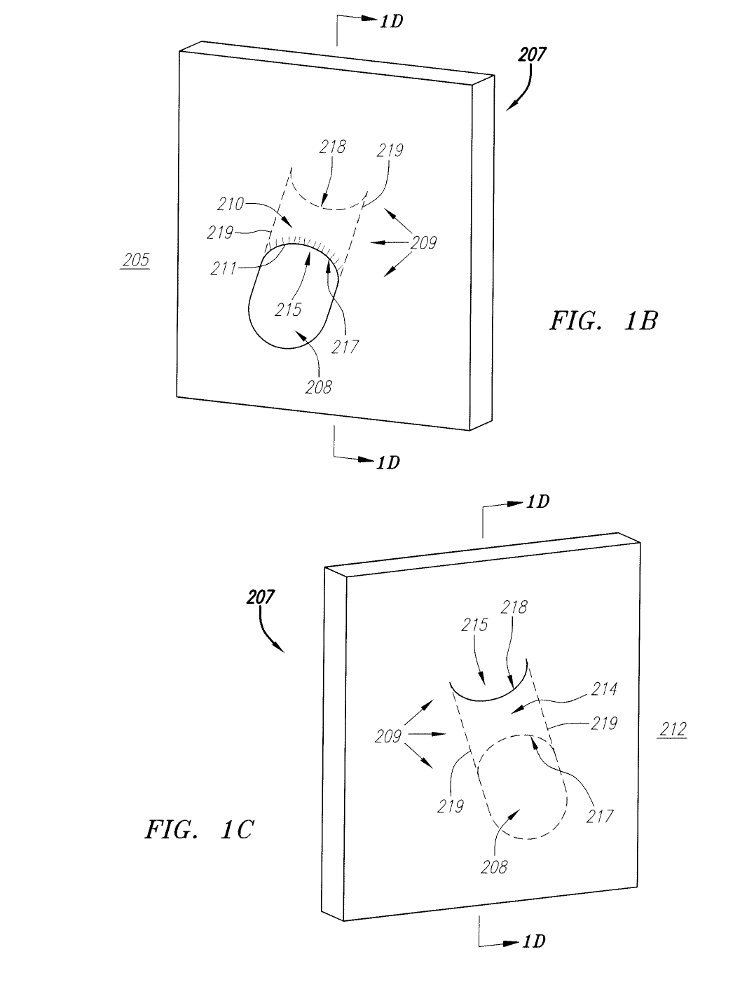

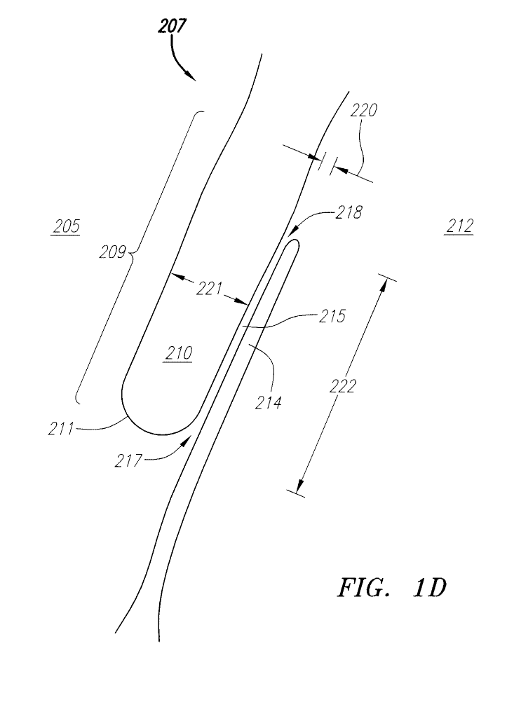

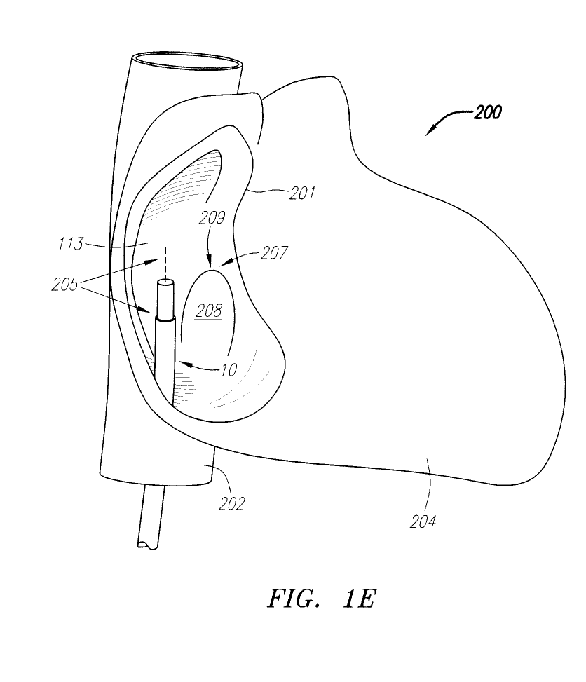

To ease the description of the many alternative embodiments of the systems and methods described herein, the anatomical structure of an example human heart having a PFO will be described in brief. FIG. 1A is an exterior/interior view depicting an example human heart 200 with a portion of the IVC 202 and the SVC 203 connected thereto. Outer tissue surface 204 of heart 200 is shown along with the interior of right atrium 205 via cutaway portion 201. Depicted within right atrium 205 is septal wall 207, which is placed between right atrium 205 and the left atrium located on the opposite side (not shown). Also depicted is fossa ovalis 208, which is a region of septal wall 207 having tissue that is relatively thinner than the surrounding tissue. PFO region 209 is located beyond the upper portion of the fossa ovalis 208.

FIG. 1B is an enlarged view of septal wall 207 depicting PFO region 209 in more detail as viewed from right atrium 205. PFO region 209 includes septum secundum 210, which is a first flap-like portion of septal wall 207. The edge of this flap above fossa ovalis 208 is referred to as the limbus 211. FIG. 1C is also an enlarged view of septal wall 207, instead depicting septal wall 207 as viewed from left atrium 212. Here, PFO region 209 is seen to include septum primum 214, which is a second flap-like portion of septal wall 207. Septum primum 214 and septum secundum 210 partially overlap each other and define a tunnel-like opening 215 between sidewalls 219 (indicated as dashed lines in FIGS. 1B-C) that can allow blood to shunt between right atrium 205 and left atrium 212 and is commonly referred to as a PFO.

FIG. 1D is a cross-sectional view depicting an example PFO region 209 taken along line 1D-1D of FIGS. 1B-C. Here, it can be seen that septum secundum 210 is thicker than septum primum 214. Typically, the blood pressure within left atrium 212 is higher than that within right atrium 205 and tunnel 215 remains sealed. However, under some circumstances, conditions can occur when the blood pressure within right atrium 205 becomes higher than the blood pressure within left atrium 212 and blood shunts from right atrium 205 to left atrium 212 (e.g., a valsalva condition). Because most typical shunts occur in this manner and for purposes of facilitating the discussion herein, region 217 in FIG. 1D will be referred to as PFO entrance 217, and region 218 will be referred to as PFO exit 218.

Many different variations of PFO's can occur. For instance, thickness 220 of septum primum 214, thickness 221 of septum secundum 210, overlap distance 222 and the flexibility and distensibility of both septum primum 214 and septum secundum 210 can all vary. In FIGS. 1B-C, PFO entrance 217 and PFO exit 218 are depicted as being relatively the same size with the width of tunnel 215, or the distance between sidewalls 219, remaining relatively constant. However, in some cases PFO entrance 217 can be larger than PFO exit 218, resulting in an tunnel 215 that converges as blood passes through. Conversely, PFO entrance 217 can be smaller than PFO exit 218, resulting in an opening that diverges as blood passes through. Furthermore, multiple PFO exits 218 can be present, with one or more individual tunnels 215 therebetween. Also, in FIGS. 1B-D, both septum primum 214 and septum secundum 210 are depicted as relatively planar tissue flaps, but in some cases one or both of septum primum 214 and septum secundum 210 can have folded, non-planar, or highly irregular shapes.

FIG. 1E depicts an exemplary catheter apparatus 10 after advancement into right atrium 205 of heart 200 through inferior vena cava 202. Here, apparatus 10 is to be used to deliver an implantable closure apparatus (not shown) from the distal end of the catheter to a PFO 209 existing in septal wall 207, superior to fossa ovalis 208. To facilitate delivery in this manner, longitudinal axis 113 of catheter 10 is preferably transverse to the plane generally defining the surface of septal wall 207 into which implant 103 is to be delivered. However, as shown in FIG. 1E, longitudinal axis 113 of catheter 10 is close to parallel to this plane. To accommodate for this, the systems, devices and methods described herein are preferably configured to allow the orientation of the delivery catheter to be changed so that the longitudinal axis 113 is transverse to this septal wall plane, which can facilitate the deployment of the implantable closure device from the catheter's distal end.

The systems, devices and methods described herein are preferably configured to treat PFOs by the application of an implantable closure apparatus deployable from a catheter, generally from within an internal lumen of a tissue piercing member or from the external surface of that member. Any closure apparatus that is deployable from a catheter can be used. To facilitate the description herein, these systems, devices and methods will be described with respect to an implantable clip-type apparatus such as those described in U.S. patent application Ser. Nos. 11/295,338 entitled “Clip-Based Systems and Methods for Treating Septal Defects,” filed Dec. 5, 2005, 11/427,572 entitled “Systems And Methods For Treating Septal Defects,” filed Jun. 29, 2006, and 11/744,784 entitled “Systems And Methods For Treating Septal Defects,” filed May 4, 2007, and U.S. Pat. No. 6,702,835 entitled “Needle Apparatus for Closing Septal Defects and Methods of Using Such Apparatus” and U.S. Pat. No. 6,776,784 entitled “Clip Apparatus for Closing Septal Defects and Methods of Use,” each of which are fully incorporated by reference herein. It should be noted, however, that the systems, devices and methods described herein are not limited solely to the application of this type of implantable device. Furthermore, if a clip similar to those described is implemented, that clip can be configured with or without a central expandable (and/or compressible) section.

Various methods of treating a PFO are described in the above-incorporated applications. However, for ease of discussion, the systems, devices and methods discussed herein will be done so with regard to a catheter-based device routed through the IVC into the right atrium of the heart. A transseptal piercing is performed from the right atrium to the left atrium (“right-to-left”), typically through both the secundum and primum. It should be noted that the systems, devices and methods can also be used when approaching from the SVC into the right atrium, in left-to-right procedures, and in procedures that involve the piercing of either the primum, secundum or both (in either order).

Described herein are various exemplary embodiments of systems, devices and methods for delivering implantable closure devices to a PFO. Some of these exemplary embodiments will be referred to as having “off-axis” delivery capability. “Off-axis” as used herein, refers to, in a device having two or more adjacent elongate members each having a longitudinal axis, the orientation of the longitudinal axis of one of the elongate members at least substantially transverse, and preferably close to perpendicular, to the longitudinal axis of the other elongate member.

Related systems and methods for treating septal defects, some of which are configured to enter an off-axis position, as well as supporting devices and methods for facilitating treatment, such as pushers, body members, and proximal controllers and the like, which can be used in conjunction with those systems, devices and methods set forth herein, are described in one or more of co-pending U.S. patent application Ser. Nos. 11/175,814 entitled “Systems and Methods for Treating Septal Defects,” filed Jul. 5, 2005 and the aforementioned incorporated '572 and '784 applications.

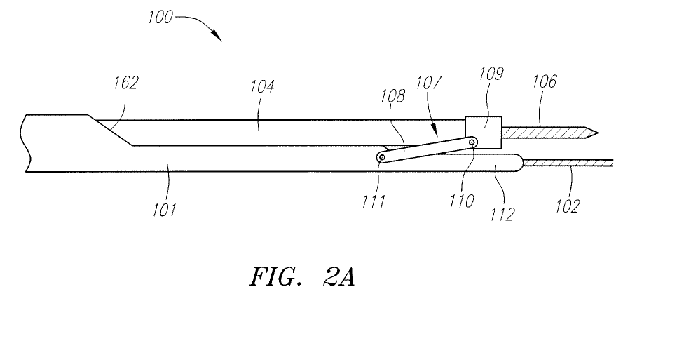

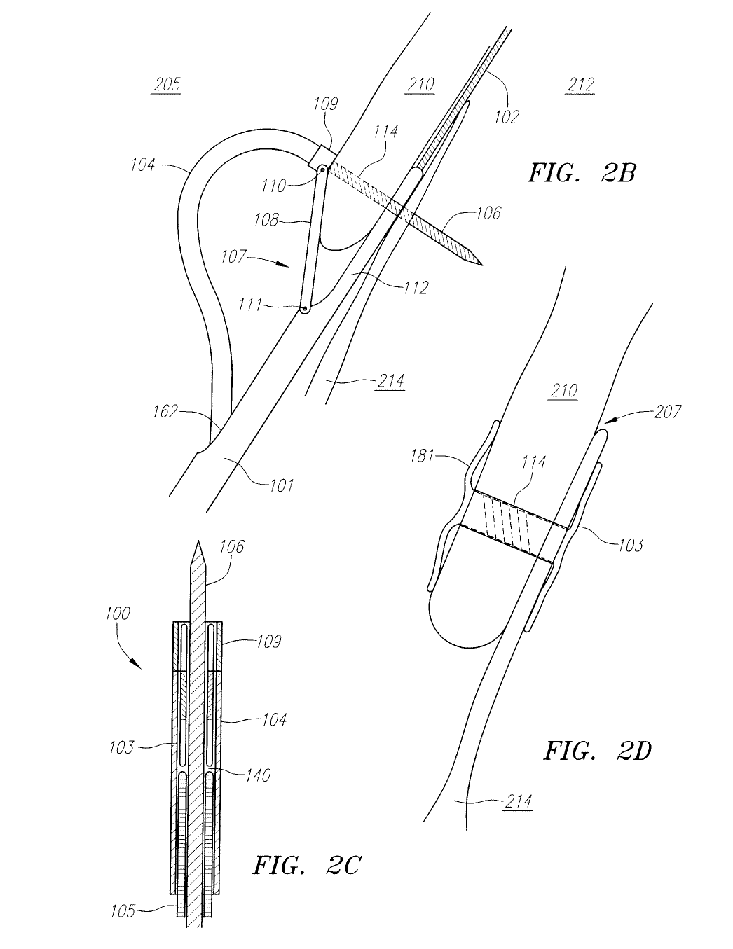

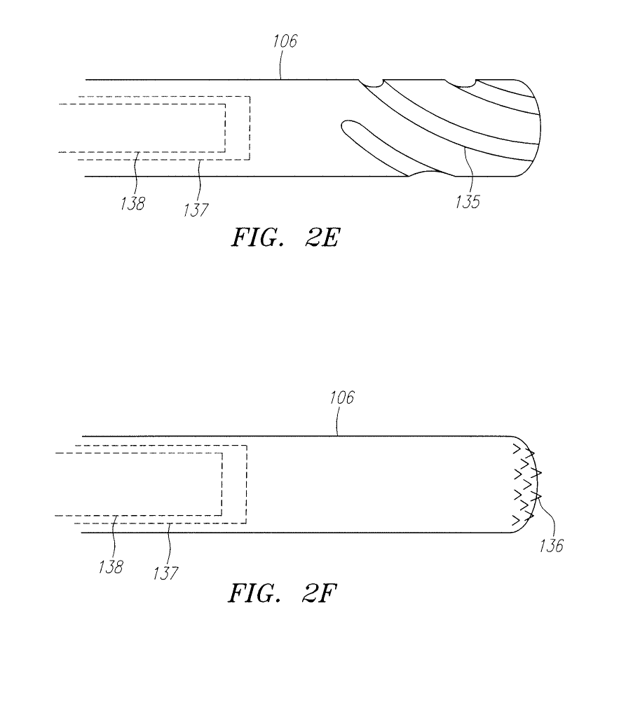

Turning now to the exemplary embodiments described herein, FIGS. 2A-C depict an exemplary embodiment of delivery system 100 configured for off-axis delivery of an implant 103 (not shown). FIG. 2A is a side view depicting system 100 in an unexpanded configuration. Here, system 100 includes an elongate body member 101 and an elongate off-axis (OA) delivery member 104 slidably disposed within a lumen (not shown) in body member 101. A skive 162 in body member 101 allows the exit of OA member 104 from body member 101. The position of skive 162 can influence or control the degree of curvature of OA member 104 when in the off-axis configuration. OA delivery member 104 is coupled with body member 101 by way of a tissue engagement device 107. OA member 104 is preferably a relatively flexible member and can include a rigid distal tip 109 for coupling with engagement device 107.

In this embodiment, the tissue engagement device 107 includes a pivotable arm 108 which is coupled with the body member 101 and rigid distal tip 109 of OA delivery member 104 at opposite ends thereof. Arm member 108, which will be generally referred to as upper jaw 108 herein, can be coupled with OA delivery member 104 and body member 101 by way of hinges 110 and 111, respectively. Hinges 110 and 111 are shown as being pin/hole type hinges, but any type of hinge with any number of one (e.g., living hinge) or more members can be used.

Body member 101 can include a distal elongate support structure 112, which will be referred to in this context as lower jaw 112. Lower jaw 112 preferably opposes at least a portion of upper jaw 108 and can be used in the engagement of septal wall 207 (e.g., septum secundum). The tissue-contacting surfaces of upper and lower jaws 108 and 112 can be textured or shaped to facilitate the latching or grasping of the septal tissue. One or more mechanical stops can be used to limit the upward retraction of upper jaw 108 when opening the jaws prior to capturing the tissue. Also, one or both of lower jaw 112 and upper jaw 108 can be offset to allow wire-like piercing member 106 to pass unimpeded. Also, lower jaw 112 can have an aperture through which member 106 can pass.

Body member 101 can also include a lumen (not shown), in which a guidewire 102 can be slidably housed. Guidewire 102 can be advanced through the patient's vasculature to guide the subsequent advancement of body member 101. In one embodiment, guidewire 102 can be advanced from the right atrium through the PFO tunnel and into the left atrium to guide system 100 into proximity with the PFO. In another embodiment, a fixed guidewire can be attached to the distal end of body member 101, so that body member 101 can be guided through the vasculature without the aid of an additional guidewire and receiving lumen.

OA delivery member 104 also includes a lumen (not shown) that can be configured to slidably receive a tissue-piercing wire-like member 106, which is shown here in a partially extended position although, in operation, member 106 is preferably kept housed within OA delivery member 104 to minimize potential harm to the patient. Although shown with a solid, pointed tissue piercing tip, other tip configurations can be used (e.g., hollow needle tip and the like).

Tissue-piercing wire-like member 106 is preferably a solid or tubular member with a tissue-piercing capability, which can derive from a mechanical piercing feature (e.g., blade, sharp point, drill-like tip and the like), electrical energy, radio frequency (RF) energy, ultrasonic energy, thermal energy or any combination thereof. Unlike a conventional needle or trocar, wire-like member 106 is characterized by its relatively reduced profile and more flexible nature. The profile of the wire can resemble that used in low profile guidewires used in neurological, cardiovascular and other highly constrained intravascular applications. In one exemplary embodiment, the outer diameter (OD) of wire-like member 106 can be as low as between 0.010″ to 0.1″ and preferably 0.014″ to 0.060.″ This OD can persist along the length of wire-like member 106, or it can be greater on the proximal portion to maintain adequate pushability (e.g., 1-to-1) and resist buckling, while at the same time maintaining flexibility so as not to significantly detract from the overall flexibility of OA member 104.

If the wire-like piercing member 106 is configured to pierce the septal tissue (e.g., either or both of the secundum and primum), member 106 preferably has sufficient rigidity at its distal end to allow this, preferably in the portion of the distal end that is advanced outside of OA member 104. Relatively more rigidity is preferable for an application that requires piercing the secundum, since this tissue is thicker and can be more difficult to penetrate than the primum.

As mentioned above, wire-like member 106 can include a piercing feature to penetrate tissue. The piercing feature can use forward movement, vibrational movement, rotational movement, different forms of energy and combinations thereof to effectuate piercing. Given the very small OD's that member 106 can have, the piercing feature may not require a relatively sharp surface, as a rounded surface can be sufficient. However, any feature that pierces tissue can be used, including a pointed tip, or a blade-like surface and the like. In one embodiment, member 106 has a drill-like tip and is configured to be rotated to facilitate penetration the septal tissue. FIGS. 2E-F are side views of two exemplary embodiments of member 106 having a plurality of blades 135 and an abrasive surface 136, respectively. The use of rotation to penetrate the tissue can reduce the rigidity requirements of member 106, since the resulting torque and drill tip will facilitate passage through the tissue such that a uni-directional pushing force does not have to be solely relied upon for penetration.

Wire-like member 106 can optionally include an interior lumen 137 with a removable core 138, the presence of which determines the rigidity of member 106 (see FIGS. 2E-F). For instance, member 106 can be used to penetrate the tissue with core 138 in place, and once access to the opposing atrial chamber is achieved, core 138 can be translated proximally to increase the flexibility of member 106 where it is absent and reduce the risk of inadvertent trauma to the surrounding atrial chamber.

Wire-like member 106 can be composed of any desired material that provides the desired operating characteristics. Exemplary materials include Nickel Titanium (NiTi) alloys, all of which are typically referred to under the generic name “NITINOL,” 304 V stainless steel and other stainless steel alloys and the like. With the use of a NITINOL alloy, member 106 can be configured with a predisposed bias towards a particular shape, which can also be thermally activated (i.e., shape memory).

Member 106 can be coated to facilitate piercing of the tissue and/or to minimize friction with any adjacent structures (e.g., implant 103, member 104). For instance, a polytetrafluoroethylene (PTFE) coating can be used to minimize friction with member 104 or implant 103. Other examples of lubricious coatings can include hydrogels (e.g., polyethylene oxide (PEO), polyvinylpyrrolidone (PVP), etc.), silicone and the like. Based on this disclosure, one of skill in the art will readily recognize the other lubricious coatings that can be used.

Wire-like member 106 can also include one or more visibility enhancing markers, such as radiopaque (e.g., Pd, Ir, Pt, Au, Ta and the like) markers to enhance visibility during fluoroscopy, as well as echogenic coatings and the like.

Unless noted otherwise, wire-like member 106 can be used with any of the embodiments described herein to create the piercing in the septal tissue.

FIG. 2B is a partial cross-sectional view of system 100 during use to treat a PFO. Here, tissue engagement device 107 is engaged with septum secundum 210 to provide a fixed reference point for treatment. Engagement with secundum 210 in this manner is described in detail in the co-pending, incorporated '814, '572 and '784 patent applications. FIG. 2B shows wire-like piercing member 106 after having been deployed from within OA delivery member 104 and through secundum 210 and primum 214 to create transseptal piercing 114.

FIG. 2C is a cross-sectional view depicting OA delivery member 104 having inner lumen 140 in which wire-like piercing member 106 is slidably housed. In this embodiment, implant 103 has a tubular configuration and is, in turn, slidably disposed about wire-like piercing member 106. Implant 103 is preferably delivered by advancing a pusher member 105 distally against implant 103. Implantation in this manner is described in the co-pending, incorporated '814, '338, '572 and '784 patent applications. To provide increased control over implant 103, pusher member 105 can be configured to engage with implant 103. Examples of pusher members configured with this capability are also described in the co-pending, incorporated '814, '338, '572 and '784 patent applications.

It should also be noted that any of the delivery techniques described herein can be used to deliver a tissue-piercing implant, such as those described in U.S. Pat. Nos. 6,776,784 and 6,702,835, both with Richard Ginn listed as the inventor, and both incorporated by reference herein in their entirety.

FIG. 2D is a cross-sectional view depicting implant 103 after implantation within septal wall 207. In this embodiment, implant 103 includes deflectable arms 181 that, once deployed from member 104, deflect outwards to anchor implant 103 and maintain secundum 210 and primum 214 in close proximity to at least partially close the PFO. An optional central coiled section 114 can be included, e.g., to provide flexibility.

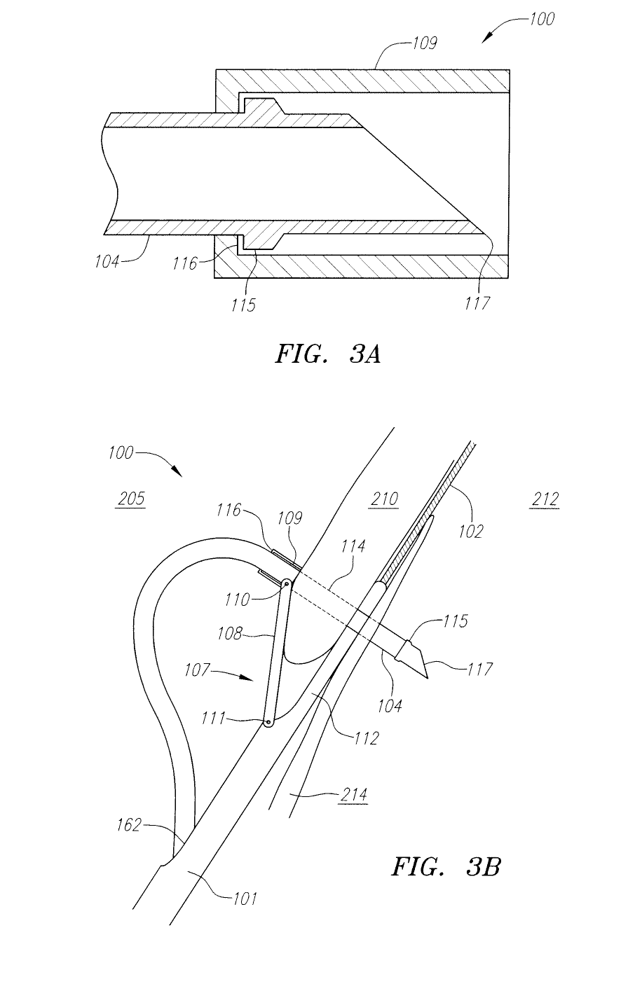

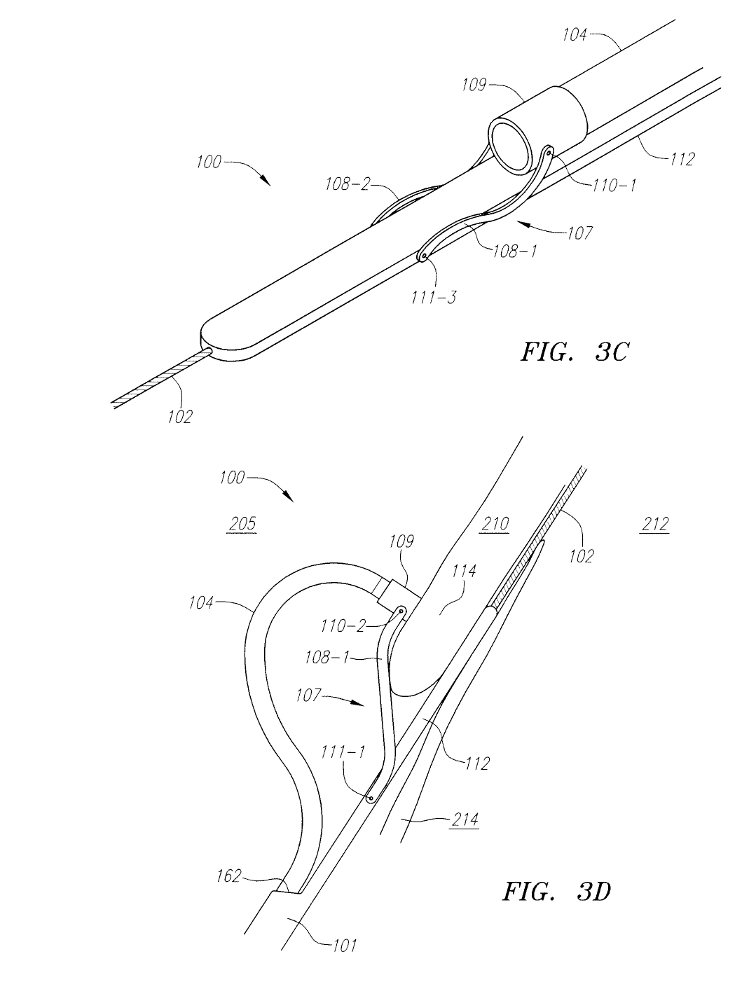

FIGS. 3A-B depict another exemplary embodiment of system 100 configured for off-axis delivery. In this embodiment, piercing 114 is created using OA deliver member 104. FIG. 3A is a cross-sectional view depicting the distal portion of OA delivery member 104, including distal tip 109. Here, OA delivery member 104 is slidably disposed within distal tip 109. OA delivery member 104 has a substantially sharp, needle-like tip 117, which is preferably housed within rigid distal portion 109 during advancement through the patient's vasculature. Needle tip 117 can be used in place of a separate needle or sharp guidewire to pierce the septal tissue. Distal tip 109 has a proximal abutment 116 that contacts a proximal abutment 115 of needle tip 117 that prevents needle tip 117 from being retracted proximally out of distal portion 109. The distal end of distal portion 109 is open to allow advancement of OA delivery member 104 there from.

FIG. 3B is a partial cross-sectional view depicting the creation of piercing 114 by OA delivery member 104. After piercing the septal tissue, implant 103 can be advanced from within OA delivery member 104 and deployed across the PFO. This embodiment eliminates the need for a needle or other tissue piercing member to be carried within OA delivery member 104, which can improve the flexibility of OA member 104 and reduce the overall profile of system 100.

FIG. 3C is a perspective view of another exemplary embodiment of system 100 configured for off-axis delivery. Here, distal tip 109 of OA delivery member 104 is pivotally coupled with two arm members, or upper jaws 108-1 and 108-2. This embodiment illustrates an alternate starting position from which OA delivery member 104 can be advanced to capture secundum 210 and subsequently enter the off-axis position. Upper jaw members 108 can be pivoted approximately 180° from the proximal position of FIG. 3C to the tissue capture/off-axis position of the partial cross-sectional view of FIG. 3D. This embodiment allows for capture of septum secundum 210 and/or entry into the off-axis position by way of one single distally-directed motion, which, among other things, can add simplicity to the deployment process.

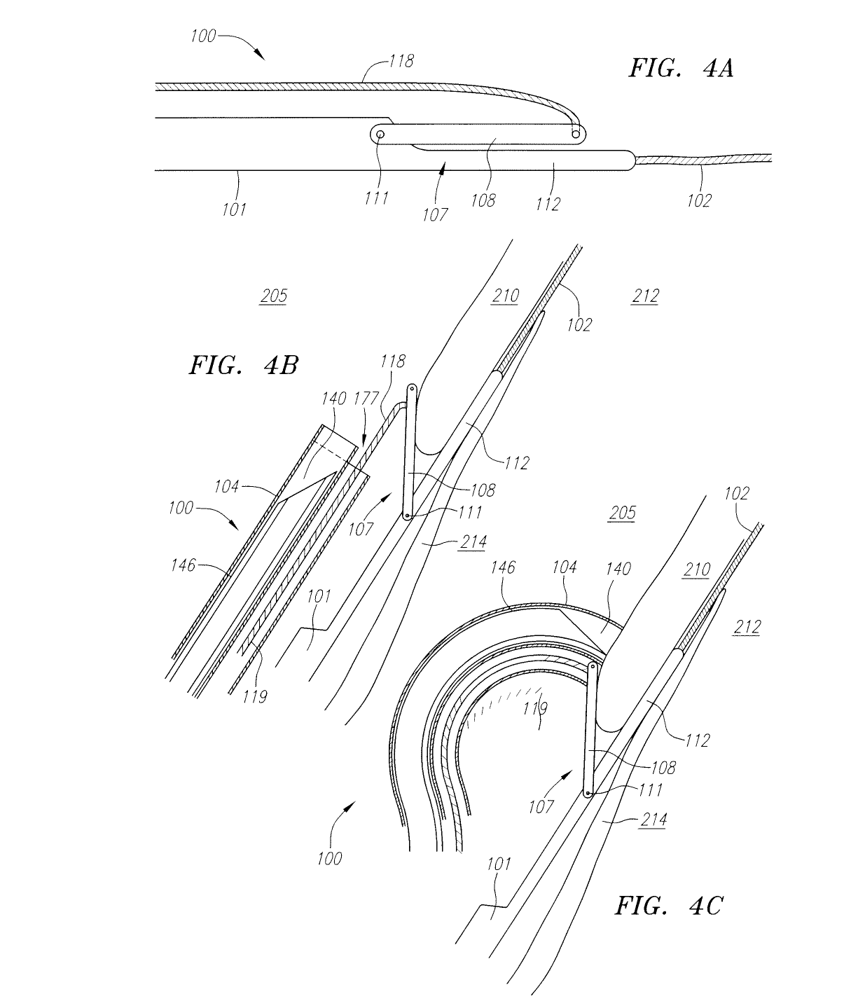

FIGS. 4A-B depict another exemplary embodiment of system 100 configured for off-axis delivery. In this embodiment, system 100 is configured to allow advancement of OA delivery member 104 (see FIG. 4B) into proximity with the PFO subsequent to the advancement of body member 101 (which is preferably advanced over guidewire 102). FIG. 4A is a side view depicting this exemplary embodiment. A pull wire 118 is coupled with the distal portion of upper jaw 108 and can be used to retract upper jaw 108 to open tissue engagement device 107 to allow it to engage septum secundum 210 as depicted in the partial cross-sectional view of FIG. 4B.

OA delivery member 104 can include a lumen 119 configured to allow OA member to be slidably advanced over pull wire 118. Preferably, tissue engagement device 107 is in contact with septum secundum 210 prior to advancement of OA delivery member 104. Once tissue engagement device 107 is properly positioned, OA delivery member 104 can be distally advanced over pull wire 118. Once the distal end of OA delivery member 104 comes into contact with upper jaw 108, further distal advancement of OA delivery member 104 causes upper jaw 108 to more fully grasp septum secundum 210 and also induces OA delivery member 104 to enter the off-axis, arced, configuration shown in the partial cross-sectional view of FIG. 4C. From this configuration, needle member 148 (shown), sharp guidewire or other piercing device can be advanced from lumen 140 and into the septal tissue to create a piercing through which implant 103 can be deployed. To allow the distal end of OA member 104 to more uniformly contact secundum 210 and upper jaw 108 in the off-axis configuration, OA member 104 can have a stepped distal tip 177 with the portion of OA member 104 corresponding to lumen 140 being relatively more distal to the portion corresponding to lumen 119.

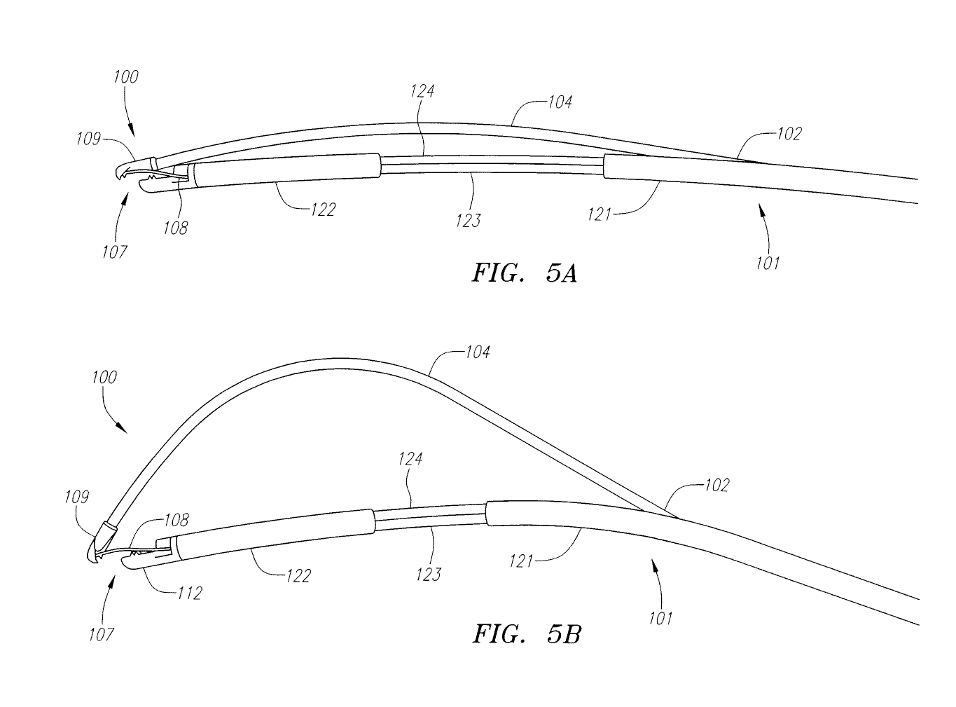

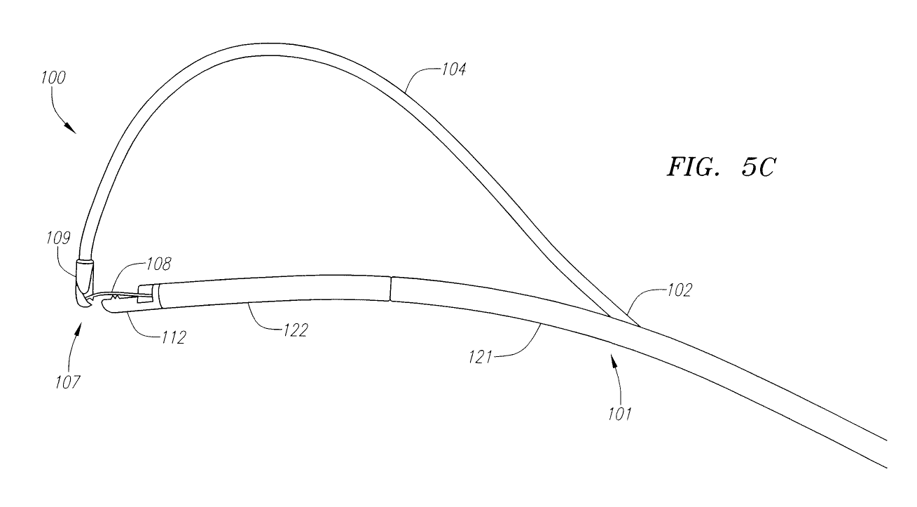

FIGS. 5A-C depict another exemplary embodiment of system 100 configured for off-axis delivery. In this embodiment, body member 101 includes proximal section 121 and distal section 122, the positions of which are adjustable relative to each other. FIG. 5A is a side view depicting this embodiment. Here, body member 101 includes sections 121 and 122 as well as one or more (in this case two) tubular inner members 123 and 124. The distal ends of tubular members 123 and 124, which are not shown, are fixably coupled with the distal end of distal section 122. Tubular inner members 123 and 124 include an open distal end through which a guidewire or other apparatus can be received. Tubular members 123 and 124 are slidably disposed within one or more lumens in proximal body section 121. Tubular members 123 and 124 can optionally include interior lumens that can be used to slidably receive a guidewire or to deploy other various devices from distal openings in body member 101.

To raise upper jaw 108, OA delivery member 104 is preferably proximally retracted with respect to body member 101, while the location of distal section 122 is maintained constant relative to proximal section 121. The position of proximal section 121 can be controlled by the user at the proximal end of system 100 by manipulation of that section while maintaining the constant relative position of tubular members 123 and 124. Proximal retraction of OA delivery member 104 will raise upper jaw 108 and allow tissue engagement device 107 to engage with the septal tissue.

Once properly positioned, proximal section 121 is preferably advanced over tubular members 123 and 124 to slide proximal section 121 towards distal section 122 of body member 101. FIG. 5B is a side view depicting proximal section 121 partially advanced towards distal section 122. The movement of proximal section 121 causes OA delivery member 104 to arc up into an off-axis delivery configuration. It also applies pressure to the distal end of arm member 108 causing tissue engagement device 107 to fasten to, or securely clamp or engage, the septal tissue.

FIG. 5C is a side view depicting proximal section 121 after being advanced into contact with distal section 122. Here OA delivery member 104 is in the fully arced position and is ready to be used to close the PFO. It should be noted that proximal section 121 is preferably moved with respect to distal section 122 when distal section 122 is engaged with the septal tissue. This can prevent inadvertent disengagement with the tissue. However, this embodiment is not limited to such and any combination of relative movement of sections 121-122 can be used as desired. Although not shown, a flexible outer sheath can be routed over the gap between sections 121-122 to maintain a uniform profile over the length of body member 101 and also reduce any seepage of bodily fluids into the interior lumen(s) of body member 101.

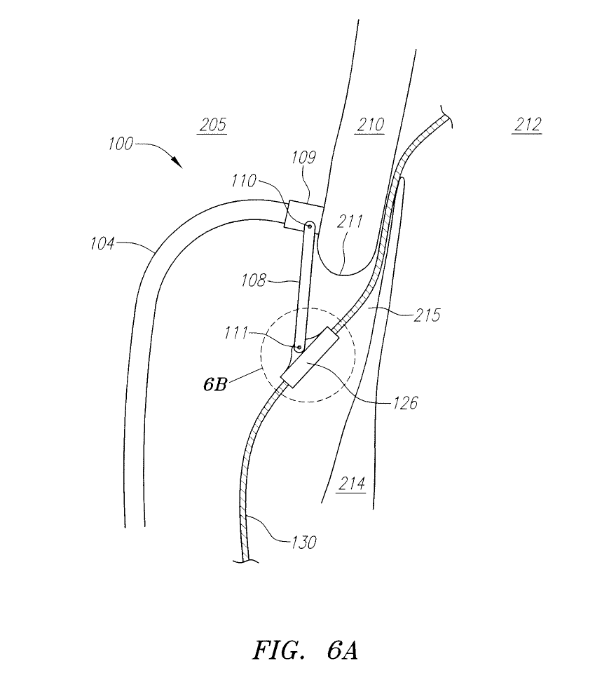

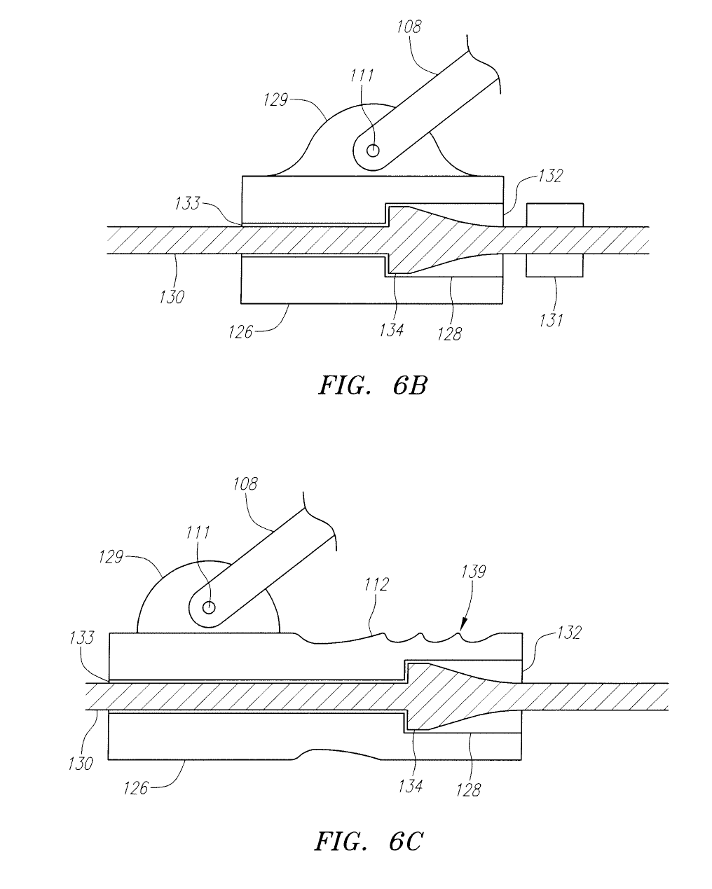

FIG. 6A is a partial cross-sectional view depicting another exemplary embodiment of system 100 configured for off-axis delivery as part of a treatment procedure. In this embodiment, system 100 is configured with a slidable guidewire anchor 126 to which upper jaw 108 is pivotally attached by way of hinge 111. FIG. 6B is a cross-sectional view depicting region 6B of FIG. 6A in more detail.

FIG. 6B depicts slidable guide wire anchor 126 with greater detail. Here, it can be seen that anchor 126 includes an inner stepped lumen 128 having a relatively wider distal opening 132 than proximal opening 133. Stepped lumen 128 includes a stop configured to interface with a relatively larger portion 134 of guidewire 130 having a complementary surface. Anchor 126 further includes a support structure 129 to which upper jaw 108 is coupled through hinge 111. A radiopaque marker 131 is also shown coupled with guidewire 130 to enhance visibility. In an alternative embodiment, marker 131 can act as the relatively larger portion 134 for stopping movement of guidewire 130 with respect to anchor 126.

Referring back to FIG. 6A, guidewire 130 is preferably advanced through PFO tunnel 215 from right atrium 205 to left atrium 212. OA delivery member 104 and slidable anchor 126 are then advanced into right atrium 205 by sliding anchor 126 distally along guide wire 130. Enlarged portion 134 of guidewire 130 is preferably visible to an external imaging system. For instance, portion 134 can be radiopaque for detection through fluoroscopy. With this visibility, the user preferably positions guidewire 130 such that enlarged portion 134 is located just proximal to limbus 211 of septum secundum 210.

Advancement of OA member 104 and corresponding anchor 126 stops once surface 128 of anchor 126 comes into contact with the opposing surface of guidewire portion 134. The positioning of portion 134 just proximal to limbus 211 ensures that anchor 126 will be in an optimum position for continuing with the procedure. Continued distal advancement of OA member 104 with respect to anchor 126 and guidewire 130 preferably causes OA delivery member 104 to swing up and outwards such that upper jaw 108 and distal tip 109 engage secundum 210, as depicted in FIG. 6A. From there, the treatment can continue in a manner similar to that described above and in the co-pending incorporated applications. OA delivery member 104 can also be configured to include one of more lumens (not shown) located proximal of the portion depicted in FIG. 6A. These lumens can be slidably advanced over guide wire 130 and can stabilize the proximal portion of OA delivery 104 with respect to guide wire 130.

FIG. 6C is a partial cross-sectional view depicting another exemplary embodiment where anchor 126 is configured to perform the functionality of lower jaw 112. Here, the upper surface of anchor 126 includes a texture 139 to facilitate gripping the septal tissue between lower jaw 112 and upper jaw 108.

It should be noted that, although complimentary stepped surfaces 128 and 131 are used to stop advancement of anchor 126 in the embodiments described with respect to FIGS. 6A-C, any surface interface can be used. One of skill in the art will readily recognize the various surfaces that can be used to stop movement. Furthermore, the surfaces can be configured to lock anchor 126 in place on guidewire 130, so as to resist movement in both the distal and proximal directions with respect to guidewire 130. An example of such a surface can be a detent-like structure on guidewire 130, that snaps into a receiving aperture in anchor 126, or vice-versa. Such interlocking surfaces can be configured to allow detachment upon the application of adequate force, or can be configured to maintain the lock and allow member 104, anchor 126 and guidewire 130 to be withdrawn as one unit.

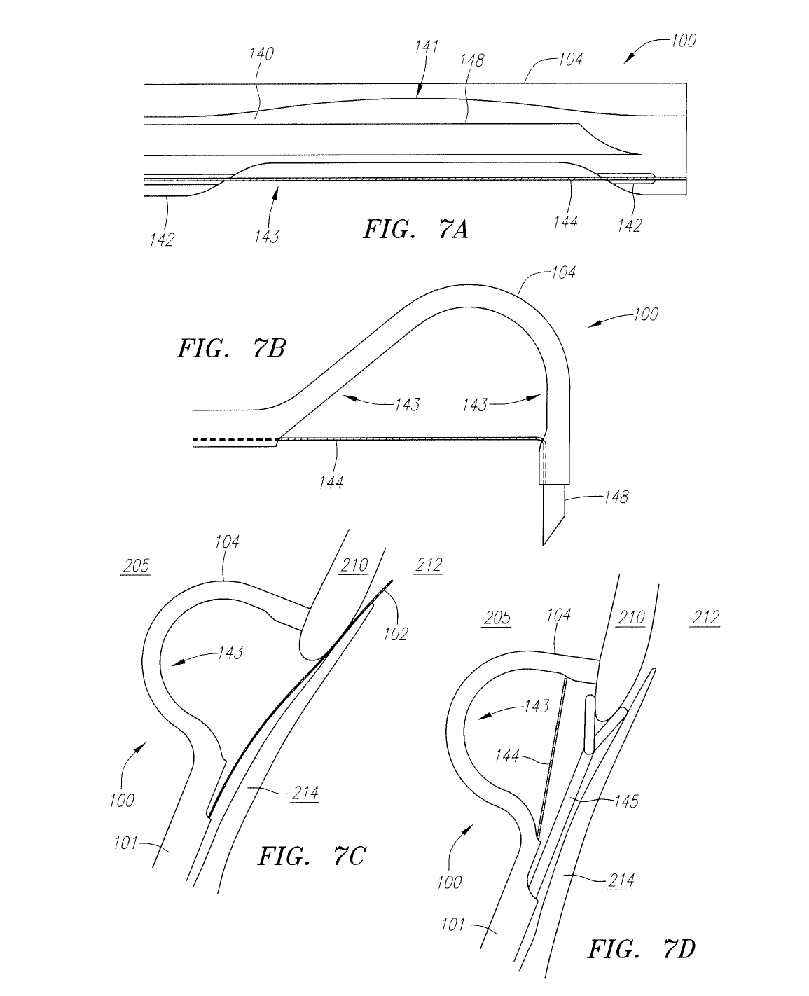

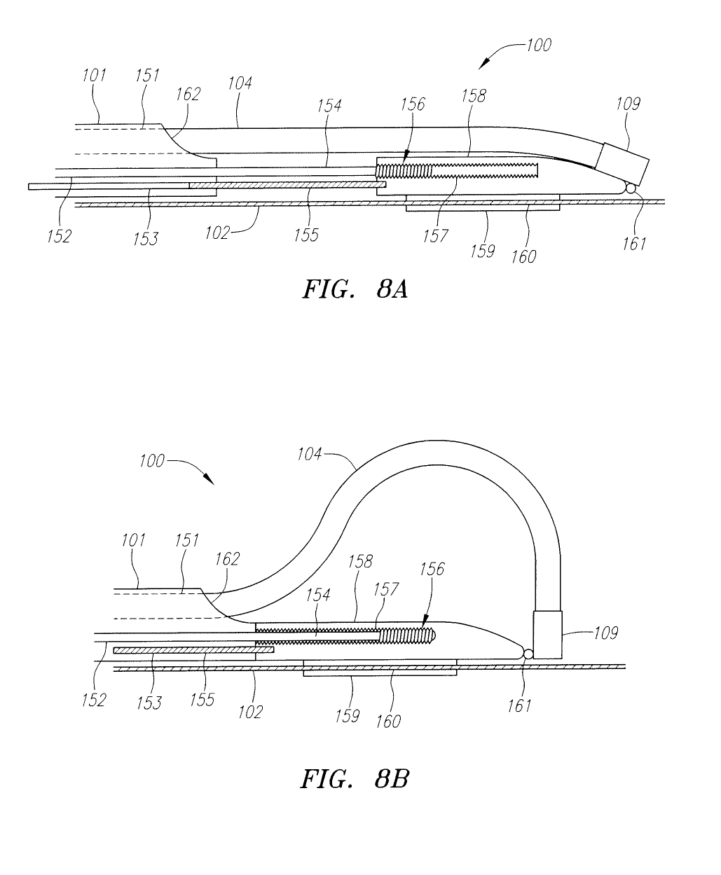

FIGS. 7A-D depict additional exemplary embodiments of system 100. FIG. 7A is a cross-sectional view of OA delivery member 104. Here, OA delivery member 104 includes lumens 140 and 142. Lumen 140 is preferably configured to slidably receive the tissue piercing member, which can be a needle-like member 148, as shown here, a sharp guide wire-like member as discussed previously or other tissue piercing device.

Lumen 142 preferably houses a pull wire 144, the distal end of which is fixably coupled with the distal end of OA delivery member 104. OA delivery member 104 also includes an open section 143 located near its distal tip through which pull wire 144 can extend. When pull wire 144 is pulled proximally as depicted in the side view of FIG. 7B, the ends of open section 143 are drawn together and OA delivery member 104 preferably enters the off-axis configuration. From this configuration, needle member 148 can be advanced relative to OA delivery member 104 to pierce through the septal tissue (not shown).

Referring back to FIG. 7A, lumen 140 can include a relatively larger width section 141 where the sidewall of OA delivery member 104 is relatively thinner, making member 104 more likely to curve or bend in the region opposite open section 143 when compressed by pull wire 144. OA delivery member 104 can also be pre-curved, or biased to curve, in region 143. One of skill in the art will readily recognize numerous different methods and techniques by which OA delivery member 104 can be configured to facilitate curvature in the desired manner when in the off-axis configuration.

Furthermore, this embodiment of OA delivery member 104 can be used in any desired manner for engaging and treating the PFO. For instance, FIG. 7C depicts a partial cross-sectional view of this embodiment of OA delivery member 104 where body member 101 and OA delivery member 104 are one monolithic structure, body member 101 includes a lumen (not shown) for advancement over guide wire 102. A similar configuration is depicted in FIG. 7D. In this embodiment, a grasping device 145 is used to engage secundum 210. This embodiment can also incorporate a guidewire 102, if desired, which can be used to guide advancement of grasping device 145 using a lumen located therein.

FIGS. 8A-B are cross-sectional views depicting another exemplary embodiment of system 100. In this embodiment, system 100 is configured to enter the off-axis configuration using a screw-type action. FIG. 8A shows this embodiment of system 100 in the low-profile configuration suitable for advancement through the patient's vasculature. Shown here is lumen 151 for slidably receiving OA delivery member 104 within body member 101. Body member 101 also includes a lumen 152 for slidably and rotatably receiving screw member 154. Screw member 154 can include a threaded distal section 156 configured to interface with a complementary threaded lumen 157 in a distal body section 158. A guide pin 155 is preferably fixably coupled with one of distal body section 158 and body member 101, in this instance, pin 155 is coupled with section 158. The proximal end of pin 155 is preferably slidably received within a lumen 153 in body member 101. Distal section 158 includes a guide wire receiving section 159 having a lumen 160 configured to slidably configure guide wire 102. Section 158 also includes a hinge 161 located at its distal tip. Hinge 161 pivotally couples section 158 with distal tip 109 of OA delivery member 104.

To cause OA delivery member 104 to enter the off-axis configuration, as depicted in FIG. 8B, screw member 154 is preferably rotated to advance threaded section 156 distally within threaded lumen 157 of section 158. Continued rotation of screw member 154 causes distal body section 158 and body member 101 to approach each other, preferably causing distal body section 158 to move proximally. The approach can continue until the end of threaded lumen 157 is reached or until section 158 makes physical contact with body member 101. Preferably, the user is provided with feedback (e.g., through internal imaging, external guides or otherwise) such that the user is aware when OA delivery member 104 reaches the off-axis configuration. This feedback can be provided by reaching a maximum displacement of section 158 or by visual indicators at the proximal end of system 100 or in any other manner desired. Furthermore, this embodiment of system 100 can be configured to operate with a tissue grasping device or other means of engaging secundum 210 to provide added stability to the insertion of clip 103 (not shown).

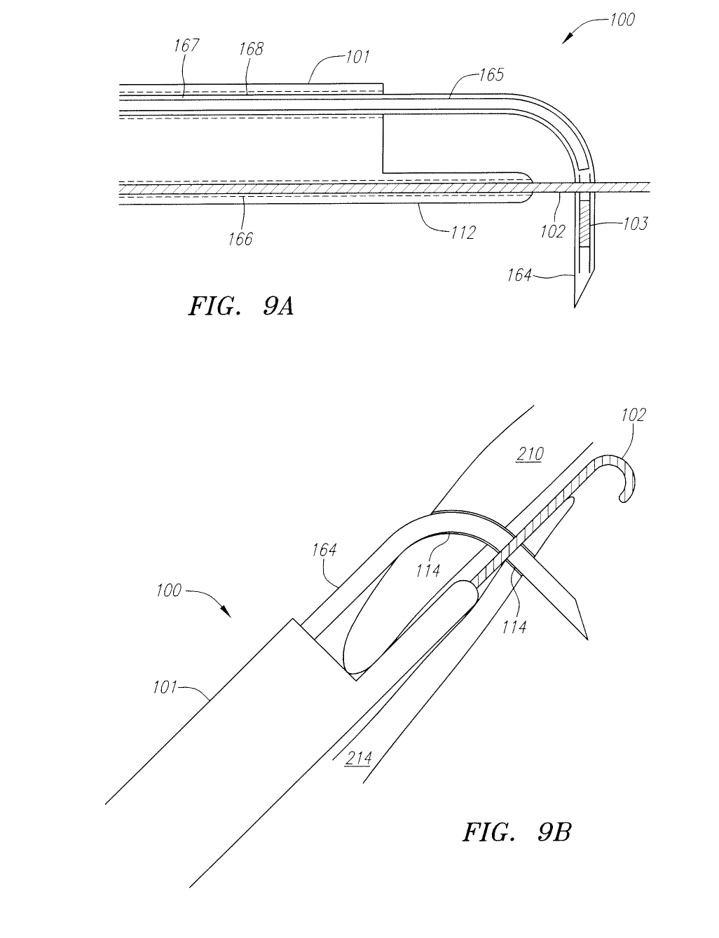

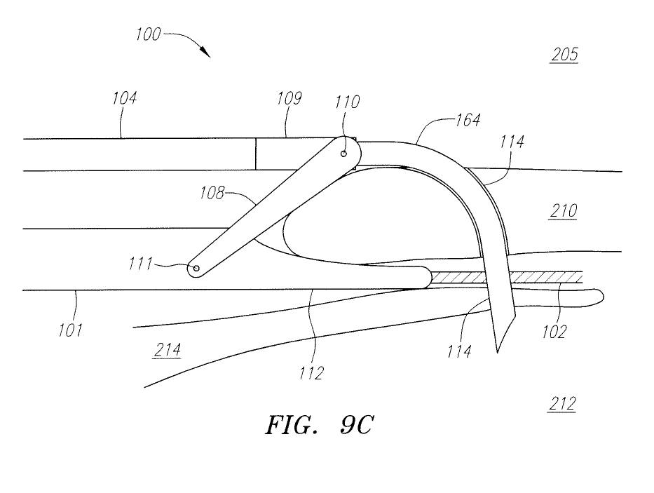

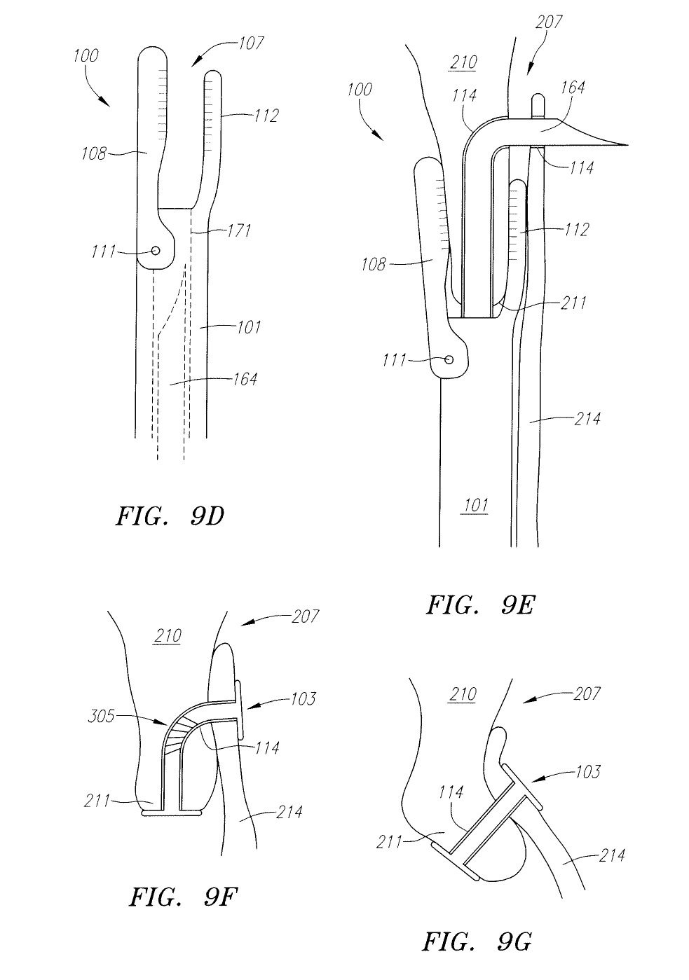

FIGS. 9A-B depict another exemplary embodiment of system 100. In this embodiment, system 100 does not include a separate OA delivery member 104. Instead, body member 101 includes lumen 168 configured to slidably receive needle member 164, as depicted in the cross-sectional view of FIG. 9A. Here, needle member 164 is shown in a deployed position. Needle member 164 is preferably biased to curve such that distal advancement of needle member 164 from within lumen 168 allows needle member 164 to penetrate the septal tissue along a generally perpendicular path and create piercing 114. For this and other embodiments herein calling for a predisposed bias, any material that can maintain a bias, such as a NITINOL alloy (e.g., by forming shape via heat treatment or using shape memory properties) or stainless steel, can be used.

Once in position, clip 103 can be advanced through piercing 114 from within lumen 167 of needle member 164 by way of pusher member 165. In this embodiment, body member 101 also preferably includes lumen 166, which is configured to slidably receive guide wire 102. Body member 101 can also include elongate support structure 112, which is configured to be inserted between secundum 210 and primum 214 as depicted in FIG. 9B. Here, needle member 164 is shown after having penetrated secundum 210 and primum 214.

Needle member 164 can generally be biased to deflect between 60 and 120 degrees, although any amount of deflection can be used. Needle member 164 is preferably biased to deflect approximately 90 degrees as shown in FIG. 9B. A deflection of “approximately 90 degrees,” is intended to include deflections that are close to but not equal to 90 degrees. Based on this disclosure, one of skill in the art will recognize that the angle at which the body member resides with respect to the septal wall will contribute to the amount of deflection desired to achieve a piercing 114 generally perpendicular to the plane of the septal wall, although in some cases, piercings that are offset from perpendicular may be desired, as in the case of a relatively short or long PFO tunnel.

FIG. 9C is a partial cross-sectional view depicting another exemplary embodiment of system 100. In this embodiment, OA delivery member 104 is a relatively rigid tubular structure. OA delivery member 104 is configured to slidably receive needle member 164, which is biased to enter a curved configuration as shown. Here, secundum 210 can be engaged in a typical manner by first proximally retracting member 104 to move jaw 108 away from jaw 112, at which point system 100 can be distally advanced into contact with secundum 210. Once in the desired position, member 104 can be distally advanced to cause jaws 108 and 112 to clamp down and engage secundum 210. Needle member 164 can then be advanced from within member 104 allowing it to enter the curved configuration and penetrate secundum 210 and primum to create piercing 114 in which clip 103 can be deployed.

FIGS. 9D-E are partial cross-sectional views depicting another exemplary embodiment of system 100 configured for use with a needle member 164 biased to enter a curved configuration. Needle member 164 is slidably housed within lumen 171 of body member 101 (needle member 164 and lumen 171 shown with dashed lines to indicate presence within body member 101) and is preferably maintained in the relatively straight configuration by the walls of body member 101. Tissue engagement device 107 is located on the distal region of body member 101 and includes upper jaw 108, which is pivotably coupled with body member 101 by way of hinge 111, and lower jaw 112, which is rigidly located on body member 101. Upper jaw 108 can be transitioned between an open and closed configuration using any desired actuation technique, including, but not limited to, push/pull wires, gear mechanisms and the like. Lower jaw 112 can also be pivotably coupled with body member 101 if desired.

FIG. 9E depicts this embodiment after tissue engagement device 107 has engaged with septum secundum 210. Although not shown, a guidewire (received within a lumen in body member 101) or other guiding element can be used to aid in positioning system 100. Once engaged with secundum 210, needle member 164 is advanced superiorly into the tissue through limbus 211. Needle member 164 then transitions to the curved configuration and exits secundum 210 and continues through septum primum 214 to create transseptal piercing 214, as shown.

Implant 103 can then be delivered within transseptal piercing 114. FIG. 9F is a cross-sectional view depicting an exemplary embodiment of implant 103 deployed within piercing 114. Here, implant 103 has a bendable region 305 that allows the implant to deform generally to the curved shape of piercing 114. FIG. 9G is a cross-sectional view depicting another exemplary embodiment of implant 103 that is relatively rigid and forces the septal tissue to deform to accommodate the relatively straight shape of the rigid implant.

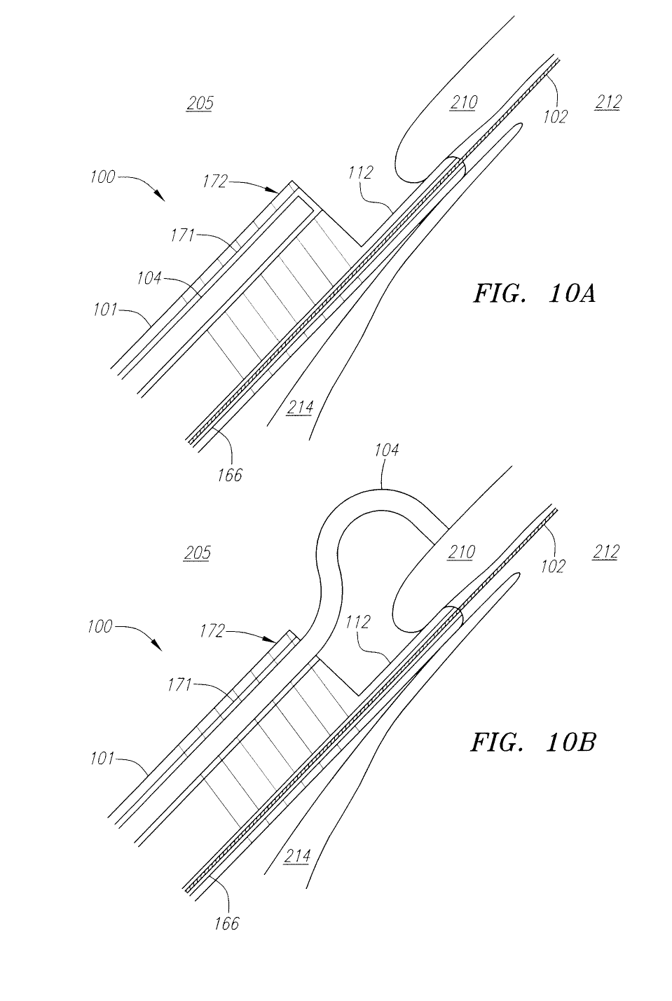

FIGS. 10A-B are partial cross-sectional views depicting an additional exemplary embodiment of system 100. In this embodiment, OA delivery member 104 is biased to enter a curved configuration once advanced from within lumen 171 of body member 101. OA delivery member 104 can be composed of a NITINOL alloy having a predisposed bias or can include a lumen into which a stylet having a predisposed bias can be advanced, the bias on the stylet being strong enough to cause OA member to deflect.

FIG. 10A shows member 104 in a relatively straight configuration within body member 101, while FIG. 10B depicts member 104 after entering the curved configuration enabled after deployment from within lumen 171. Also shown here is lumen 166 configured to receive guide wire 102. The distal portion of body member 101 having lumen 171 can be reinforced to resist the curvature of member 104 prior to deployment. In this embodiment, reinforcement 172 includes a wire wrapping over (or within) body member 101. One of skill in the art will readily recognize the numerous methods by which body member 101 can be reinforced, e.g., braided sections, stiffening bars, rigid sheaths, counteracting stylets and the like. It should also be noted that a septal tissue engagement apparatus can also be used to engage the septal tissue and stabilize the position of body member 101.

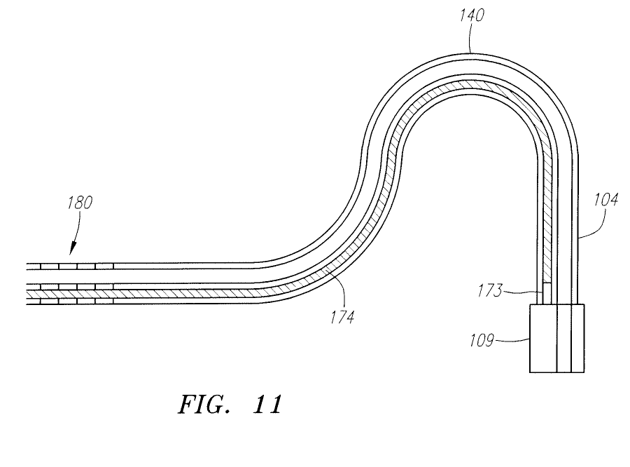

FIG. 11 is a cross-sectional view depicting another exemplary embodiment of OA delivery member 104. Here, member 104 includes lumen 140 from which a needle member, sharp guidewire or other piercing device can be advanced. Member 104 also includes a lumen 173 in which a stylet 174 can be advanced (as shown). Stylet 174 is biased to transition to a curved configuration with sufficient strength to force member 104 to adopt the corresponding shape. This occurs once stylet 174 is distally advanced within lumen 173 to a relatively more pliable or flexible section of OA member 104. Here, member 104 includes a reinforcement 180 along its proximal length, making the distal portion of member 104 relatively more flexible than the proximal reinforced portion. In this configuration, distal advancement of stylet 174 past reinforcement 180 will allow stylet 174 to enter the curved configuration and, likewise, cause member 104 to enter the curved configuration shown here. Again, one of skill in the art will readily recognize the numerous different ways in which member 104 can be reinforced. Of course, either or both of OA member 104 and a needle member can also be curved or biased to curve to facilitate the transition to the curved state described with respect to FIG. 11.

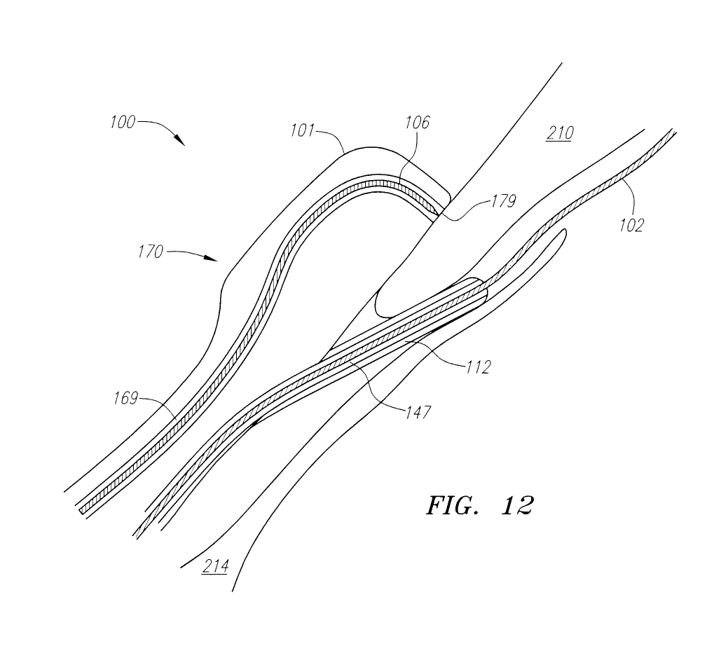

FIG. 12 is a partial cross-sectional view of another exemplary embodiment of system 100. Here, body member 101 includes a curved lumen 169 configured to slidably receive wire-like piercing member 106. Lumen 169 is preferably oriented within body member 101 to guide piercing member 106 out of lumen distal end 179 at an orientation suitable for piercing member to penetrate septum secundum 210 and (optionally) septum primum 214. In this embodiment, the outer diameter of body member 101 is kept sufficiently small to allow flexing through the patient's vasculature over the desired route. In order to reduce the friction created in passing member 106 through the curved portion of lumen 169, a relatively large radius of curvature for lumen 169 can be desirable. To provide for a relatively larger radius of curvature while at the same time allowing for a relatively small outer diameter, body member 101 can be optionally configured with a bulb-like distal portion 170 as depicted here. This bulb-like distal portion 170 has a relatively larger outer diameter than the proximal portion of body member 101 to provide greater space in which lumen 169 can be routed.

In this embodiment, although not shown, the implant can be delivered with a pusher member in a manner similar to that described with respect to FIG. 2C. In an alternative embodiment, after creation of the piercing through the septal tissue, the piercing member 106 can be withdrawn and the implant inserted into lumen 169 and delivered to the piercing with the aid of a pusher member. In another alternative embodiment, once the piercing member 106 has penetrated the septal tissue, e.g., through both secundum 210 and primum 214, body member 101 can be withdrawn leaving piercing member 106 in place. An implant can then be advanced over piercing member 106 and deployed. Alternatively, prior to withdrawal of body member 101, piercing member 106 can be withdrawn and replaced with a guidewire that extends through the piercing created by member 106. Body member 101 can then be withdrawn and an implant can be advanced over the guidewire and deployed.

In the embodiment depicted in FIG. 12, lower jaw 112 is present and preferably used to engage secundum 210. Lower jaw 112 can be opened with the use of a pull wire or push wire (not shown) or any other actuating means known to one of skill in the art. Here, lower jaw 112 and body member 101 include a lumen 149 configured to slidably receive guidewire 102, which is preferably routed through the PFO first to allow it to guide body member 101 into position. In another embodiment, lower jaw 112 can be omitted such that guidewire 102 exits the lumen 149 in body member 101 directly. This configuration can help automatically guide body member 101 into the appropriate position with respect to secundum 210.

The devices and methods herein may be used in any part of the body, in order to treat a variety of disease states. Of particular interest are applications within hollow organs including but not limited to the heart and blood vessels (arterial and venous), lungs and air passageways, digestive organs (esophagus, stomach, intestines, biliary tree, etc.). The devices and methods will also find use within the genitourinary tract in such areas as the bladder, urethra, ureters, and other areas.

Other locations in which and around which the subject devices and methods find use include the liver, spleen, pancreas and kidney. Any thoracic, abdominal, pelvic, or intravascular location falls within the scope of this description.

The devices and methods may also be used in any region of the body in which it is desirable to appose tissues. This may be useful for causing apposition of the skin or its layers (dermis, epidermis, etc), fascia, muscle, peritoneum, and the like. For example, the subject devices may be used after laparoscopic and/or thoracoscopic procedures to close trocar defects, thus minimizing the likelihood of subsequent hernias. Alternatively, devices that can be used to tighten or lock sutures may find use in various laparoscopic or thoracoscopic procedures where knot tying is required, such as bariatric procedures (gastric bypass and the like) and Nissen fundoplication. The subject devices and methods may also be used to close vascular access sites (either percutaneous, or cut-down). These examples are not meant to be limiting.

The devices and methods can also be used to apply various patch-like or non-patchlike implants (including but not limited to Dacron, Marlex, surgical meshes, and other synthetic and non-synthetic materials) to desired locations. For example, the subject devices may be used to apply mesh to facilitate closure of hernias during open, minimally invasive, laparoscopic, and preperitoneal surgical hernia repairs.

It should be noted that various embodiments are described herein with reference to one or more numerical values. These numerical value(s) are intended as examples only and in no way should be construed as limiting the subject matter recited in any claim, absent express recitation of a numerical value in that claim.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure.

What is claimed is:

1. A method for treating a patent foramen ovate, comprising:
advancing a guidewire through the inferior vena cava, between the septum secundum and septum primum and into the left atrium of a patient;
advancing a treatment apparatus along the guidewire such that the distal end of a lumen of the apparatus is in proximity with the septum secundum; and
advancing a tissue-piercing member from within the lumen and through the septum secundum, wherein the tissue-piercing member is biased to deflect from a first, relatively straight configuration to a second, relatively curved configuration upon advancement from within the lumen.

2. The method of claim 1, further comprising deploying an implantable closure apparatus from the tissue-piercing member.

3. The method of claim 2, wherein the lumen is a first lumen and the treatment apparatus comprises an elongate body member having a second lumen configured to guide advancement of the apparatus over the guidewire.

4. The method of claim 3, wherein the body member includes the first lumen.

5. The method of claim 3, wherein the apparatus further comprises an elongate delivery member including the first lumen.

6. The method of claim 5, wherein the apparatus further comprises a tissue engagement device.

7. The method of claim 6, wherein the tissue engagement device is configured to engage the septum secundum.

8. The method of claim 7, wherein the tissue engagement device comprises an arm member pivotally coupled between the body member and the delivery member.

9. The method of claim 8, wherein the delivery member comprises a relatively rigid distal portion configured to resist the bias of the tissue-piercing member.

10. The method of claim 8, wherein the body member comprises a distal elongate support structure configured to oppose the arm member and engage the septal tissue therebetween.

11. The method of claim 8, wherein the delivery member is slidably disposed within the body member.

12. The method of claim 4, wherein the body member comprises a distally located portion having relatively more rigidity than the adjacent portion of the body member.

13. The method of claim 12, wherein the distally located portion is a reinforced portion.

14. The method of claim 13, wherein the reinforced portion comprises braids.

15. The method of claim 13, wherein the reinforced portion comprises a wire wrapping.

16. The method of claim 1, further comprising grasping the septum secundum prior to advancing the tissue-piercing member.

17. The method of claim 1, wherein the tissue-piercing member is biased to deflect between 60 and 120 degrees.

18. The method of claim 17, wherein the tissue-piercing member is biased to deflect about 90 degrees.

19. The method of claim 1, further comprising piercing the septum primum after piercing the secundum.

20. The method of claim 1, further comprising deploying a clip through the secundum and primum, the clip having a right atrial anchor and a left atrial anchor.

21. The method of claim 1, wherein the tissue-piercing member is advanced superiorly through the limbus of the septum secundum.
